(19) United States
(12) Patent Application Publication
(10) Pub. No.: US 2026/0013753 A1
(43) Pub. Date: Jan. 15, 2026

(54) BODY MOUNT FOR MOUNTING AT LEAST ONE TRANSDERMAL MEDICAL DEVICE TO A BODY SURFACE OF A USER

(71) Applicant: Roche Diabetes Care, Inc., Indianapolis (IN)

(72) Inventor: Kirill Sliozberg, Mannheim (DE)

(21) Appl. No.: 19/331,099

(22) Filed: Sep. 17, 2025

Related U.S. Application Data
(63) Continuation of application No. PCT/EP2024/058066, filed on Mar. 26, 2024.

(30) Foreign Application Priority Data
Mar. 29, 2023 (EP) 23165175.3

(51) Int. Cl. A61B 5/145; A61B 5/00

(52) U.S. Cl. CPC A61B 5/14532; A61B 5/14503; A61B 5/6833; A61B 5/6848; A61B 5/7271; A61B 2560/0468; A61B 2562/0214

(57) ABSTRACT

A body mount for mounting at least one transdermal medical device to a body surface of a user is disclosed. The body mount includes: at least one base element having at least one mounting surface for attachment to the body surface of the user, at least one capillary for taking up blood from the body of the user when the base element is attached to the body surface of the user; and at least one bleeding detector configured for detecting blood in the capillary. Further disclosed are a medical device including the body mount, a medical kit including the medical device, a method of monitoring a transdermal medical device, and a method of determining at least one physiological parameter in a body tissue of a user and computer programs and computer-readable storage media for performing the methods.

### CROSS REFERENCE TO RELATED APPLICATIONS

This is a continuation of application serial no. PCT/EP2024/058066 filed Mar. 26, 2024 which claims priority from EP 23 165 175.3 filed Mar. 29, 2023.

### BACKGROUND

### Technical Field

The present disclosure relates to a body mount for mounting at least one transdermal medical device to a body surface of a user, to a medical device and a medical kit. The disclosure further relates to a method of monitoring a transdermal medical device and to a method of determining at least one physiological parameter in a body tissue of a user and to computer programs and computer-storage media for performing the methods. The devices and the methods may be applied in the field of continuous monitoring of, e.g., analytes in bodily fluids of the user, specifically in the field of home care and in the field of professional care, such as in hospitals. Other applications, however, are also feasible.

### Background Art

Monitoring certain body functions, more particularly monitoring one or more analyte concentrations such as one or more metabolite concentration in a body fluid of a user plays an important role in the prevention and treatment of various diseases. Such analytes can include by way of example, but not exclusively, glucose, lactate, cholesterol or other types of analytes and metabolites. Without restricting further possible applications, the invention will be described in the following text with reference to glucose monitoring. However, additionally or alternatively, the invention can also be applied to other types of analytes, such as the analytes mentioned above, and/or other physiological parameters.

Generally, systems for continuous glucose monitoring (CGM) in a body tissue of a user are known. For example, WO 2016/016217 A1 discloses a medical sensor assembly comprising a flexible plaster adapted for adhesion on the skin of a patient and a sensor configured for transdermal measuring a physiological parameter. The sensor has a measuring part insertable into the skin and a contact part for providing a signal connection to a measuring unit. The plaster has a flap which is adhered to an intermediate section of the sensor between the measuring part and the contact part.

U.S. Pat. No. 8,864,664B2 describes a medical device for carrying out at least one medical function. The medical device comprises at least one control part, which can be applied to a body surface of a user, and at least one functional element, which can be inserted into a body tissue of the user at at least one insertion site. The functional element can be connected to the control part. The functional element is designed to carry out at least one medical function. The control part has at least one base part with at least one supporting surface, which faces the body surface. The base part has at least one collection channel for collecting bodily fluid emerging from the insertion site.

EP3771400A1 discloses a physiological signal monitoring device including a base, a biosensor, a transmitter, and a scaling unit. The biosensor includes a mounting seat and a sensing member that is mounted to the mounting seat. The sensing member is adapted to be partially inserted underneath the skin surface for measuring an analyte of the host and to send a corresponding physiological signal. The transmitter is for receiving and transmitting the physiological signal, and has a bottom portion. The transmitter covers the base while the bottom portion faces the base. The sensing member is coupled to the transmitter. The sealing unit is used to seal paths through which a liquid possibly penetrates into an interior of the physiological signal monitoring device so as to avoid damage to the device.

Despite the advantages achieved by the known devices and methods, several technical challenges remain. Specifically, CGM systems may be strongly influenced by bleeding caused by insertion of the GGM sensor into the body tissue of the user, specifically during first hours or even days after the insertion of the CGM sensor. For example, substances comprised by blood, e.g., thrombocytes, may be adsorpted on a surface of the CGM sensor and, thus, may block glucose diffusion towards a sensing layer within the CGM sensor. This may cause a temporary reduced sensor sensitivity. Thus, the measured glucose values may be lower than the real blood glucose value. Depending on the severity of the bleeding, the duration of the signal distortion may amount to several hours or even up to several days, which specifically depends on the decomposition rate of the built deposit by the body. Therefore, CGM sensors which generally comprise a factory-calibrated sensor may underestimate the glucose values indicated to the user. Further, some CGM systems perform an initial calibration. In this case, if bleeding occurs, the CGM sensor sensitivity may be calculated based on a diminished initial glucose signal. Later on, once the deposit around the CGM sensor may be decomposed by the body, the sensor sensitivity generally increases. However, the initial calibration calculated sensitivity may still be considered for CGM signal generation. This may result in overestimated glucose levels which is highly dangerous for the user of the CGM system. Thus, there is a need to for CGM sensors to detect bleeding and consider its appearance.

### SUMMARY

It is desirable to provide devices and methods which at least partially address above-mentioned technical challenges. Specifically, devices and methods shall be proposed which allow detecting a presence and/or a quantity of bleeding upon insertion, i.e., during and/or after insertion, of transdermal medical devices into a body tissue of a user.

This problem is addressed by a body mount for mounting at least one transdermal medical device to a body surface of a user, by a medical device, by a medical kit, by a method of monitoring a transdermal medical device and by a method of determining at least one physiological parameter in a body tissue of a user as well as by computer programs and computer-readable storage media for performing the methods. Advantageous embodiments which might be realized in an isolated fashion or in any arbitrary combinations are described throughout the specification.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once.

Further, as used in the following, the terms “preferably”, “more preferably”, “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

In a first aspect of the present disclosure, a body mount for mounting at least one transdermal medical device to a body surface of a user is disclosed.

The term “body mount” as used herein is a broad term and is to be given its ordinary and customary meaning to a person of ordinary skill in the art and is not to be limited to a special or customized meaning. The term specifically may refer, without limitation, to a device or combination of devices being configured for attachment to a body of a user. The body mount may specifically be configured for being temporarily attached to a body of the user, such as by generating or using a removable connection with the body of the user. The body of the user, as outlined in further detail below, may be a body of a living being, such as a human being or an animal, and, thus, a connection between the body mount and the body of the user may be biocompatible, such as by using biocompatible adhesive materials or the like, which may have as little detrimental effects on the user or the body of the user as possible, at least during typical durations of use. The body mount may be part of a medical device, as will be outlined in further detail below, and, thus, may be configured for interacting with one or more further components of the medical device. For example, the body mount may be configured for supporting, carrying, interacting and/or being connected with the one or more further devices, such as at least one transdermal medical device and/or at least one evaluation unit. Thus, as an example, the body mount may be configured to act as a holder, holding one or more components, such as one or more medical components, and/or for directly or indirectly attaching these one or more components to a body surface of the user. For example, the body mount may be configured for interacting with an insertion aid for least partially inserting the transdermal medical device. For example, the body mount may be an integral part of the medical device, such as by being a component of an integral patch of the medical device, e.g. forming an integral element with the medical device. Alternatively, the body mount may be a separate part of the medical device, such as a body mount being handled independently from the medical device. However, the body mount may be configured for functionally and/or structurally interacting with the medical device in order to provide at least one common function. For example, the body mount may be provided with electric connection means for electrically connecting the body mount and the medical device. The body mount may specifically provide a sensor patch, specifically by interaction with the transdermal medical device and/or the evaluation unit. For attachment to the body surface of the user, the body mount may comprise, as will be discussed in further detail below, one or more adhesive components and/or one or more adhesive surfaces. Additionally, or alternatively, other types of attachment, however, are also feasible.

The term “mounting” as used herein is a broad term and is to be given its ordinary and customary meaning to a person of ordinary skill in the art and is not to be limited to a special or customized meaning. The term specifically may refer, without limitation, to a process of attaching, connecting and/or adhering two or more devices and/or objects with each other. Specifically, by mounting at least one device to at least one other device or object, a connection, in particular a removable connection, between the connected devices or objects may be formed.

The term “transdermal” as used herein is a broad term and is to be given its ordinary and customary meaning to a person of ordinary skill in the art and is not to be limited to a special or customized meaning. The term specifically may refer, without limitation, to a property of an element or device being capable of being partially inserted into and/or underneath a skin of a living being. The transdermally inserted element, in the inserted state, may be fully or partially implanted. Thus, as an example, one or more portions of the clement may protrude from a body tissue of the user, through the skin of the user, to the outside environment. Specifically, a partial insertion of the transdermal element or device may refer to a situation in which one part of the transdermal element or device is inserted into and/or underneath the skin, wherein another part of the transdermal element or device remains above the skin. Thus, a transdermal insertion of the transdermal element or device may comprise a partial implanting or positioning of the transdermal element or device into or underneath the skin of a living being. The transdermal element may be configured for remaining in the inserted portion, such as for several hours, more specifically for one or more days, ever more specifically for up to two weeks or even more.

The term “medical device” as used herein is a broad term and is to be given its ordinary and customary meaning to a person of ordinary skill in the art and is not to be limited to a special or customized meaning. The term specifically may refer, without limitation, to an arbitrary clement or article being configured for use in the field of medical technology, specifically in the field of medical analytics or medical diagnostics. The medical device may be configured for performing at least one medical function and/or for being used in at least one medical process, such as one or more of a therapeutic process, a diagnostic process or another medical process. Consequently, the term “transdermal medical device” as used herein is a broad term and is to be given its ordinary and customary meaning to a person of ordinary skill in the art and is not to be limited to a special or customized meaning. The term specifically may refer, without limitation, to a medical device, i.e. to an arbitrary clement or article being configured for use in the field of medical technology, which is capable of being partially inserted into and/or underneath a skin of a living being. The part of the transdermal medical device which is inserted into and/or underneath the skin may be referred to as “intracorporal part”, whereas the part of the transdermal medical device which remains above the skin may be referred to as “extracorporal part”.

The term “body surface” as used herein is a broad term and is to be given its ordinary and customary meaning to a person of ordinary skill in the art and is not to be limited to a special or customized meaning. The term specifically may refer, without limitation, to an exterior and/or upper boundary of the user's body. As outlined in further detail below, the user may be a living being, such as a human being or an animal, and, thus, the body surface may be defined by a skin of the human being or animal. The body surface may specifically comprise a skin of the living being.

The term “user” as used herein is a broad term and is to be given its ordinary and customary meaning to a person of ordinary skill in the art and is not to be limited to a special or customized meaning. The term specifically may refer, without limitation, to a living being, such as a human being or an animal, independent from the fact that the living being may be in a healthy condition or may suffer from one or more diseases. As an example, the user may be a patient suffering from diabetes. However, additionally or alternatively, the present disclosure may be applied to other types of users, patients or diseases. The user may be the living being, such as a human being or an animal, to which the body mount is applied to. The user may be different from a person handling the body mount. The body mount may be applied to the user by a medically trained person examining the user which, as an example is suffering from diabetes and/or other diseases, specifically by a medical healthcare person, such as in point-of-care applications. The body mount may also be applied by the user himself or herself, specifically in homecare applications.

The body mount comprises:
at least one base element having at least one mounting surface for attachment to the body surface of the user,
at least one capillary for taking up blood from the body of the user when the base element is attached to the body surface of the user;
at least one bleeding detector configured for detecting blood in the capillary.

The term “base element” as used herein is a broad term and is to be given its ordinary and customary meaning to a person of ordinary skill in the art and is not to be limited to a special or customized meaning. The term specifically may refer, without limitation, to a device providing at least one supporting function. The base element may be configured for providing support for one or more elements of the body mount, such as the transdermal medical device, one or more detectors and/or one or more control units. Specifically, the base element may provide support for the one or more elements of the body mount such that the base element together with the one or more supported elements of the body mount may be attachable, specifically as a unit, to the body surface of the user. The base element may be configured for being attached to the body surface of the user, specifically to a skin of the user. Specifically, the base clement may comprise one or more components which, alone or in combination, provide the attaching function of the base element. One of these components may be the mounting surface of the base element. The one or more components which provide the attaching function, as an example and as outlined above and in further detail below, may comprise one or more adhesive surfaces and/or one or more adhesive elements.

The term “mounting surface” as used herein is a broad term and is to be given its ordinary and customary meaning to a person of ordinary skill in the art and is not to be limited to a special or customized meaning. The term specifically may refer, without limitation, to an clement forming at least one interface with the body surface of the user. Specifically, the mounting surface may provide at least one contacting area, wherein, in the contacting area, the body surface of the user and the mounting surface may be in direct contact. The mounting surface may be one or more of a flat surface or a curved surface. The mounting surface may be configured, for example by being made of a flexible material, for adapting to the body surface of the user. The mounting surface may be or may comprise the one or more adhesive surfaces and/or the one or more adhesive elements as discussed above and as outlined in further detail below.

The base element may at least partially be made of at least one flexible material. Additionally, or alternatively, the base clement may be at least partially made of at least one plaster, specifically at least one adhesive plaster, more specifically at least one flexible plaster adapted for adhesion on the skin of the user. Specifically, the mounting surface may comprise at least one adhesive for adhesion to the body surface of the user.

The term “capillary” as used herein is a broad term and is to be given its ordinary and customary meaning to a person of ordinary skill in the art and is not to be limited to a special or customized meaning. The term specifically may refer, without limitation, to an arbitrary element forming at least one cavity configured for receiving fluids, specifically bodily fluid, such as blood or any blood comprising fluid. Specifically, the capillary may be a hollow element forming the at least one cavity in its interior. The capillary may be a tube, in particular a small tube, such as a tube with a limited height and/or with a limited cross section or diameter. The capillary, in particularly the tube, may be at least partially formed within the base element. The capillary may be configured for conducting the received fluid along a predetermined path. Specifically, upon entry into the capillary, the fluid may flow through the capillary, thus being transported by the capillary along the predetermined path. The transport of the fluid through the capillary may be at least partially promoted by capillary force. The capillary may be a capillary channel.

Alternatively, or additionally, the mounting surface may comprise the at least one adhesive, as outlined above, and the capillary may be formed by the adhesive. Specifically, the adhesive may comprise at least one structure for taking up blood from the body of the user thereby forming the capillary. Further, if bleeding occurs, a color of the adhesive may change due to saturation by the blood. By using a bleeding detector with reflectometry, the color change may be detected. This configuration may provide a simple setup of the bleeding detector.

The capillary may comprise at least one capillary selected from the group consisting of: a straight capillary; a curved capillary; a conical capillary; a capillary with varying profile; a broadening capillary, specifically a capillary broadening from an opening towards an end of the capillary; a capillary formed in the adhesive of the mounting surface.

The capillary may at least partially be formed within the base element, specifically integrally. The base element may at least partially be made of at least one thermoplastic polymer material, for example polycarbonate, which, specifically, may be suitable for being used in a molding manufacturing process, such as injection molding, transfer molding and/or compression molding. The capillary may, thus, be formed integrally within the base element, for example, by forming at least one recess in the base element during the molding manufacturing process.

The capillary may be coated by at least one hydrophilization layer. The hydrophilization layer may comprise at least one layer of at least one material having hydrophilic properties. Specifically, the hydrophilization layer may improve the capillary's wetting properties.

Additionally, or alternatively, the capillary may be scaled by at least one foil, specifically at least one hydrophilic foil. Thus, the capillary being sealed by the at least one foil may form a complete capillary. For example, the capillary may be at least partially formed within the base element by providing the base element with at least one recess, e.g. at least one groove. The foil may seal the recess in the base element to form the capillary.

Additionally, or alternatively, the base element may further comprise at least one through-hole allowing the transdermal medical device to at least partially extend through the base element. The through-hole may fully or at least partially be surrounded by material of the base element. In other words, the base element may form at least one circumferential rim of the through-hole or may partially form at least one rim of the through-hole. Thus, the through-hole, as an example, may be a or may comprise a hole or a slit in the base element. The capillary, specifically an opening of the capillary, may be arranged adjacent to the through-hole. Thus, in case insertion of the intracorporal part of the transdermal medical device may cause bleeding in the body tissue of the user, the opening of the capillary may allow the blood to enter the capillary.

The term “blood” as used herein is a broad term and is to be given its ordinary and customary meaning to a person of ordinary skill in the art and is not to be limited to a special or customized meaning. The term specifically may refer, without limitation, to any fluid comprising at least one constituent of blood, such as blood plasma and/or cellular constituents of blood. For example, blood may comprise whole blood, specifically whole blood as naturally present in the human or animal body. For example, blood may comprise interstitial fluid.

The term “detector” as used herein is a broad term and is to be given its ordinary and customary meaning to a person of ordinary skill in the art and is not to be limited to a special or customized meaning. The term specifically may refer, without limitation, to an arbitrary device configured for detecting, i.e. for at least one of determining, measuring and monitoring, at least one parameter, qualitatively and/or quantitatively, such as at least one of a physical parameter, a chemical parameter and a biological parameter. The detector may be configured for generating at least one detector signal, more specifically at least one electrical detector signal, such as an analogue and/or a digital detector signal, the detector signal providing information on the at least one parameter measured by the detector. Consequently, the term “bleeding detector” as used herein is a broad term and is to be given its ordinary and customary meaning to a person of ordinary skill in the art and is not to be limited to a special or customized meaning. The term specifically may refer, without limitation, to a detector for qualitatively and/or quantitatively detecting at least one parameter related to bleeding. The bleeding detector may specifically detect a presence and/or an amount of blood in the capillary. The bleeding detector may be configured for generating at least one detector signal as outlined above, the detector signal providing information on the presence and/or the amount of blood in the capillary.

The bleeding detector may be selected from the list comprising an optical bleeding detector and an electrical bleeding detector. The term “optical bleeding detector” as used herein is a broad term and is to be given its ordinary and customary meaning to a person of ordinary skill in the art and is not to be limited to a special or customized meaning. The term specifically may refer, without limitation, to a bleeding detector configured for detecting the at least one parameter related to bleeding using optical detection means. Specifically, the optical bleeding detector may use light in the optical spectral range, such as in the visible spectral range, in the infrared spectral range and/or in the ultraviolet spectral range, to detect the presence and/or the amount of blood in the capillary.

The bleeding detector may comprise at least one optical emitter element for emitting light into the capillary and at least one optical detection element for detecting light from the capillary. The optical emitter element may comprise at least one light source, such as a light emitting diode (LED), for emitting light into the capillary. The optical emitter element may specifically be configured for emitting light in a spectral range where an absorbance of blood is maximum, for example in a spectral range at least partially comprising wavelengths from 350 to 450 nm and/or wavelengths from 500 to 600 nm. However, other wavelength ranges are also feasible.

The optical detection element may comprise at least one photosensitive element configured for determining at least one optical parameter, such as an intensity and/or a power of light by which at least one sensitive area of the optical detection element is irradiated. For example, the optical detection element may comprise at least one photodiode, photocell, photosensitive resistor, phototransistor, photomultiplier or bolometer. The optical bleeding detector may comprise a plurality of optical emitter elements, specifically of LEDs, each optical emitter element being associated with at least one optical detection element, specifically at least one photodiode. For example, the optical bleeding detector may comprise a plurality of pairs of optical emitter elements and optical detection elements. Additionally, or alternatively, the optical bleeding detector may comprise a plurality of optical detection elements for one optical emitter element. The bleeding detector, specifically the optical bleeding detector, may be configured for bleeding detection by at least one of a transmissive optical measurement and a reflective optical measurement.

For example, the optical emitter element and the optical detection element may be arranged for direct illumination. The optical emitter element and the optical detection element may be arranged on opposing sides of the capillary, specifically such that light emitted by the optical emitter element may directly illuminate the capillary and, subsequently, the optical detection element, more specifically without being reflected along an illumination path. Thus, in this arrangement, the bleeding detector may be configured for transmissive optical measurement.

For example, the optical emitter element and the optical detection element may be arranged for reflective illumination. The optical emitter element and the optical detection element may be arranged on the same side of the capillary, specifically adjacent to each other, such that light emitted by the optical emitter element may illuminate the capillary, be reflected at least once at the capillary, e.g. at least once at one or more reflective capillary walls, and, subsequently, illuminate the optical detection element. The reflective illumination may specifically comprise at least one reflection of light in the illumination path. Thus, in this arrangement, the bleeding detector may be configured for reflective optical measurement.

Further, the base element may be at least partially made of at least one optically transparent material. Specifically, the capillary may be at least partially integrated into the base element and at least one wall of the capillary may be at least partially made of the optically transparent material. It may be possible to use the optical bleeding detector for detection of bleeding and, additionally, it may be possible to detect bleeding by using bare eyes.

The term “electrical bleeding detector” as used herein is a broad term and is to be given its ordinary and customary meaning to a person of ordinary skill in the art and is not to be limited to a special or customized meaning. The term specifically may refer, without limitation, to a bleeding detector configured for detecting the at least one parameter related to bleeding using electrical detection means. Specifically, the electrical bleeding detector may detect at least one electrical quantity, such as conductivity, capacitance and/or impedance, to detect the presence and/or the amount of blood in the capillary.

The bleeding detector may comprise at least one electrical bleeding detector. The electrical bleeding detector may comprise at least two electrodes. For example, the electrical bleeding detector may comprise a plurality of electrodes arranged along the capillary. Additionally, or alternatively, the electrical bleeding detector may comprise two electrodes extending along the capillary, specifically along the entire capillary. Additionally, or alternatively, in case the transdermal medical device comprises at least one transdermal sensor, at least one of the electrodes of the electrical bleeding detector may be formed by the transdermal sensor. The electrical bleeding detector may be configured for detection of blood by at least one of a conductivity measurement, a capacitive measurement and an impedance measurement.

Further, the body mount may comprise a plurality of bleeding detectors arranged along the capillary in order to detect a filling level of the capillary. Specifically, the body mount may comprise a plurality of optical bleeding detectors arranged along the capillary in order to detect a filling level of the capillary. Additionally, or alternatively, the body mount may comprise a plurality of electrical bleeding detectors having at least two electrodes arranged along the capillary in order to detect a filling level of the capillary.

The body mount may further comprise at least one control unit. The term “control unit” as used herein is a broad term and is to be given its ordinary and customary meaning to a person of ordinary skill in the art and is not to be limited to a special or customized meaning. The term specifically may refer, without limitation, to an arbitrary logic circuitry configured for performing basic operations of a computer or system, and/or, generally, to a device which is configured for performing calculations or logic operations. The control unit may specifically comprise one or more processors. In particular, the processor may be configured for processing basic instructions that drive the computer or system. As an example, the processor may comprise at least one arithmetic logic unit (ALU), at least one floating-point unit (FPU), such as a math co-processor or a numeric co-processor, a plurality of registers, specifically registers configured for supplying operands to the ALU and storing results of operations, and a memory, such as an L1 and L2 cache memory. In particular, the processor may be a multi-core processor. Specifically, the processor may be or may comprise a central processing unit (CPU).

Additionally, or alternatively, the control unit may be or may comprise a microprocessor, thus specifically the elements of the control unit may be contained in one single integrated circuitry (IC) chip. Additionally, or alternatively, the control unit may be or may comprise one or more application-specific integrated circuits (ASICs) and/or one or more field-programmable gate arrays (FPGAs) and/or one or more tensor processing unit (TPU) and/or one or more chip, such as a dedicated machine learning optimized chip, or the like. The control unit specifically may be configured, such as by software programming, for performing one or more control operations and/or evaluation operations, as will be outlined in further detail below. Specifically, the control unit may be configured, such as by software programming, for processing at least one bleeding detector signal from the bleeding detector. For example, the control unit may be configured for generating at least one item of information regarding a bleeding status, specifically at least one warning signal in case a bleeding is detected. The warning signal may comprise one or more of an electrical warning signal, e.g. provided to an evaluation unit as will be outlined in further detail below, or an optical warning signal, e.g. by using the optical emitter element. For example, an optical warning signal may be provided to the user using at least one green LED as the optical emitter element. The green LED may be arranged such that the green LED light may be visible to the user in case no bleeding occurs.

In a further aspect of the present disclosure, a medical device is disclosed. The medical device comprises at least one body mount according to the present disclosure, such as according to any one of the embodiments disclosed above and/or according to any one of the embodiments disclosed in further detail below. Thus, for definitions of terms related to and for possible embodiments of the body mount, reference is made to the description of the body mount above. The medical device further comprises at least one transdermal medical device having at least one intracorporal part being configured for transdermal insertion into a body tissue of the user and at least one extracorporal part attached to the body mount.

The term “body tissue” as used herein is a broad term and is to be given its ordinary and customary meaning to a person of ordinary skill in the art and is not to be limited to a special or customized meaning. The term specifically may refer, without limitation, to a part of the user, i.e. a part of a living being. The body tissue may comprise fatty tissue and/or interstitial tissue. The body tissue may comprise dermal tissue, such as epidermal tissue and/or dermal tissue, and/or subcutaneous tissue, specifically depending on an insertion depth of the transdermal medical device, more specifically depending on an insertion of the intracorporal part of the transdermal medical device into and/or underneath the skin. Other types of body tissue, however, are feasible. The body tissue may specifically be located underneath the body surface of the user.

The transdermal medical device specifically may be selected from the group consisting of: a transdermal sensor, specifically at least one transdermal sensor for measuring at least one physiological parameter, more specifically a transdermal analyte sensor, more specifically at least one transdermal analyte sensor for detecting at least analyte in a bodily fluid of the user; an infusion device having at least one tubular infusion element for administering at least one medically active substance into the body tissue of the user.

The term “transdermal sensor” as used herein is a broad term and is to be given its ordinary and customary meaning to a person of ordinary skill in the art and is not to be limited to a special or customized meaning. The term specifically may refer, without limitation, to a device configured for sensing, i.e. qualitatively or quantitatively determining a presence, a value and/or a concentration of at least one physiological parameter. The transdermal sensor may be configured for sensing the physiological parameter in the body tissue of the user. Thus, the transdermal sensor may be configured for transdermal insertion. The transdermal sensor may remain in the inserted body tissue, such as for several hours, specifically for one or more days, more specifically for up to one week, even more specifically for up to two weeks or even more. The transdermal sensor may specifically be configured for continuously sensing the physiological parameter in the body tissue of the user.

The term “physiological parameter” as used herein is a broad term and is to be given its ordinary and customary meaning to a person of ordinary skill in the art and is not to be limited to a special or customized meaning. The term specifically may refer, without limitation, to any determinable physiological quantity of the user, specifically of the body tissue of the user. The physiological parameter may specifically be related to bodily functions of the user. Thus, the physiological parameter may be configured for being used in medical diagnostics. The physiological parameter may comprise, e.g., a presence and/or a concentration of at least one analyte, a pulse rate, a blood pressure or the like.

The term “analyte” as used herein is a broad term and is to be given its ordinary and customary meaning to a person of ordinary skill in the art and is not to be limited to a special or customized meaning. The term specifically may refer, without limitation, to a chemical and/or biological substance which takes part in the metabolism of the body of the user. Specifically, the analyte may be a metabolite or a combination of two or more metabolites. As an example, the analyte may be selected from the group consisting of: glucose, lactate, triglycerides, cholesterol. Still, other analytes or combinations of two or more analytes may be detected.

The term “bodily fluid” as used herein is a broad term and is to be given its ordinary and customary meaning to a person of ordinary skill in the art and is not to be limited to a special or customized meaning. The term specifically may refer, without limitation, to arbitrary liquid body fluid which is present in the body tissue of the user, i.e. in the human or animal being, such as blood and/or interstitial fluid.

The term “transdermal analyte sensor” as used herein is a broad term and is to be given its ordinary and customary meaning to a person of ordinary skill in the art and is not to be limited to a special or customized meaning. The term specifically may refer, without limitation, to a transdermal sensor configured for sensing the presence and/or the concentration of at least one analyte in the bodily fluid.

The transdermal analyte sensor may be an electrochemical transdermal analyte sensor. The transdermal analyte sensor may comprise at least two electrodes. Specifically, the transdermal analyte sensor may comprise at least one two-electrode sensor. The two-electrode sensor may comprise precisely two electrodes, such as a working electrode and at least one further electrode such as a counter electrode, e.g. a working electrode and a combined counter/reference electrode. The working electrode may comprise a working electrode pad and, optionally, at least one test chemical disposed thereon. The counter electrode may comprise a counter electrode pad. Additionally, and optionally, one or more redox materials may be disposed thereon. The transdermal analyte sensor may further comprise one or more leads for electrically contacting the electrodes. The leads may, during insertion or at a later point in time, be connected to one or more electronic components, specifically to the control unit. For example, the leads may already be connected to the electronic components before insertion of the transdermal analyte sensor. For example, the transdermal analyte sensor may be a needle-shaped or a strip-shaped transdermal analyte sensor having a flexible substrate and the electrodes disposed thereon. As an example, the transdermal analyte sensor may have a total length of 5 mm to 50 mm, specifically a total length of 7 mm to 30 mm. The term “total length” within the context of the present disclosure relates to the overall length of the transdermal analyte sensor which corresponds to both the intracorporal part and the extracorporal part of the transdermal analyte sensor. For example, the intracorporal part may have a length in the range from 3 mm to 12 mm. The transdermal analyte sensor may further comprise a biocompatible cover, such as a biocompatible membrane which fully or partially covers the transdermal analyte sensor and which prevents the test chemical from migrating into the body tissue and which allows for a diffusion of the body fluid and/or the analyte to the electrodes. Other embodiments of electrochemical transdermal analyte sensors, such as three-electrode sensors, may be feasible. For example, the three-electrode sensor may comprise, in addition to the working electrode and the counter electrode, a reference electrode.

The transdermal analyte sensor may be an optical analyte sensor. For example, the transdermal analyte sensor may comprise a flexible light guide with analyte, e.g. glucose, sensitive coating at its end and/or a tube like carrier with functional elements at inner or outer walls. Other embodiments of the transdermal analyte sensor may be possible too.

The term “infusion device” as used herein is a broad term and is to be given its ordinary and customary meaning to a person of ordinary skill in the art and is not to be limited to a special or customized meaning. The term specifically may refer, without limitation, to a device or a combination of device for delivering and/or infusing a medication into the body tissue of the user. Specifically, the infusion device may comprise at least one transdermal tubular infusion element. The infusion device, as an example, may comprise at least one transdermal tubular infusion element and optionally at least one extracorporal part, such as at least one extracorporal tubular infusion element fluidically connected to the transdermal tubular infusion element, wherein the extracorporal tubular infusion element may be configured, e.g. by one or more connection elements, for being fluidically connected to a fluid reservoir.

The term “tubular infusion element” as used herein is a broad term and is to be given its ordinary and customary meaning to a person of ordinary skill in the art and is not to be limited to a special or customized meaning. The term specifically may refer, without limitation, to a hollow tube configured for delivering and/or infusing a medication into the body tissue of the user, in particular for delivering and/or infusing insulin into the body tissue of the user. The tubular infusion element may comprise at least one infusion cannula.

The term “medically active substance” as used herein is a broad term and is to be given its ordinary and customary meaning to a person of ordinary skill in the art and is not to be limited to a special or customized meaning. The term specifically may refer, without limitation, to any chemical and/or biological substance which is used in a therapeutic treatment of the metabolism of the body of the user. The medically active substance may provide the medication to the user.

The transdermal medical device may specifically comprise the at least one transdermal sensor. The medical device may further comprise at least one evaluation unit. The term “evaluation unit” as used herein is a broad term and is to be given its ordinary and customary meaning to a person of ordinary skill in the art and is not to be limited to a special or customized meaning. The term specifically may refer, without limitation, to an arbitrary logic circuitry configured for performing basic operations of a computer or system, and/or, generally, to a device which is configured for performing calculations or logic operations. The evaluation unit may specifically comprise one or more processors which, specifically, may be embodied as outlined above. The evaluation unit may specifically be partially or completely identical with the control unit, such as by being partially or completely embodied by the same logic circuitry. Additionally, or alternatively, the evaluation unit may also comprise at least one additionally logic circuitry. The evaluation unit may be configured, such as by software programming, for determining the at least one physiological parameter by evaluating the at least one detector signal provided by the transdermal sensor. The evaluation unit may further be configured, such as by software programming, for taking into account the at least one bleeding detector signal provided by the bleeding detector. The bleeding detector may be configured for directly or indirectly providing the bleeding detector signal to the evaluation unit.

The medical device specifically may be or may comprise a continuous glucose monitoring (CGM) device. The CGM device may comprise the at least one transdermal analyte sensor for detecting the at least analyte in the bodily fluid of the user, specifically for continuously detecting the analyte in the bodily fluid of the user. However, additionally or alternatively, the medical device also may be or may comprise a one-use blood glucose meter, such as for single detection of the analyte in the bodily fluid.

In a further aspect of the present disclosure, a medical kit is disclosed. The term “medical kit” as used herein is a broad term and is to be given its ordinary and customary meaning to a person of ordinary skill in the art and is not to be limited to a special or customized meaning. The term specifically may refer, without limitation, to an assembly of a plurality of components, wherein the components each may function and may be handled independently from each other. The components of the kit may interact with each other to perform at least one common function, specifically at least one medical function, and/or being used in at least one common process, specifically at least one medical process, such as one or more of a therapeutic process, a diagnostic process or another medical process. The components of the kit may be supplied conjointly in a package.

The medical kit comprises at least one medical device according to the present

disclosure, such as according to any one of the embodiments disclosed above and/or according to the embodiments disclosed in further detail below. Thus, for definitions of terms related to and for possible embodiments of the medical device and the body mount, reference is made to the description of the medical device and of the body mount above.

The medical kit further comprises at least one insertion aid for inserting the intracorporal part of the transdermal medical device into the body tissue of the user. The term “insertion aid” as used herein is a broad term and is to be given its ordinary and customary meaning to a person of ordinary skill in the art and is not to be limited to a special or customized meaning. The term specifically may refer, without limitation, to a device configured for inserting the transdermal medical device into the body tissue of the user, such as by performing an incision or a puncture in the skin of the user and by transferring at least the intracorporal part of the transdermal medical device fully or partially into the body tissue of the user. The insertion aid may be configured for transcutaneously or subcutaneously inserting the intracorporal part of the transdermal medical device into the body tissue of the user. Upon inserting the intracorporal part of the transdermal medical device into the body tissue of the user, the insertion aid may specifically be configured for attaching of the base element of the body mount to the body surface of the user.

In a further aspect of the present disclosure, a method of monitoring a transdermal medical device is disclosed. The term “monitoring” as used herein is a broad term and is to be given its ordinary and customary meaning to a person of ordinary skill in the art and is not to be limited to a special or customized meaning. The term specifically may refer, without limitation, to a process of surveying at least one parameter of at least one device, e.g. in order to identify at least one operational state of the device. The monitoring may specifically comprise continuously surveying the at least one parameter and/or surveying the at least one parameter at least once within fixed or variable time intervals. The monitoring may comprise acquiring the at least one parameter, e.g. continuously and/or at least once within fixed or variable time intervals, and deriving desired information therefrom, specifically without user interaction, such as automatically. For this purpose, at least one measurement signal, such as a plurality of measurement signals, may be generated and evaluated, wherefrom the desired information may be determined. Herein, at least one measurement signal may be recorded continuously or at least once within fixed or variable time intervals or, alternatively or in addition, at an occurrence of at least one pre-specified event. The monitoring of the medical device may specifically comprise surveying the at least one bleeding detector signal, e.g. in order to identify at least one bleeding status of the medical device, specifically comprising at least one of a presence and/or an amount of blood in the capillary of the body mount of the medical device. As an example, a result of the monitoring may comprise at least one item of information on the presence or absence of blood in the capillary. Additionally, or alternatively, a result of the monitoring may comprise a filling level of blood in the capillary.

The method comprises the following steps that, as an example, may be performed in the given order. It shall be noted, however, that a different order may generally also be possible. Further, it may also be possible to perform one or more of the method steps once or repeatedly. Further, it may also be possible to perform two or more of the method steps simultaneously or in a timely overlapping fashion. The method may comprise further method steps that are not listed.

The method comprises evaluating at least one bleeding detector signal of at least one bleeding detector of at least one medical device according to the present disclosure, such as according to any one of the embodiments disclosed above and/or according to any one of the embodiments disclosed in further detail below, for detecting blood in the least one capillary of the body mount of the medical device.

The method specifically may comprise using the medical device according to the present disclosure, such as according to any one of the embodiments disclosed above and/or according to any one of the embodiments disclosed in further detail below. For definitions of terms related to and possible embodiments of the medical device and the body mount, reference is made to the description of the medical device and the body mount above.

The body mount specifically may comprise the at least one control unit as outlined above. The method may further comprise processing the at least one bleeding detector signal from the bleeding detector by using the control unit. The processing of the bleeding detector signal may comprise generating at least one item of information regarding a bleeding status, specifically at least one warning signal in case a bleeding is detected.

The method specifically may be at least partially computer-implemented. The term “computer-implemented” as used herein is a broad term and is to be given its ordinary and customary meaning to a person of ordinary skill in the art and is not to be limited to a special or customized meaning. The term specifically may refer, without limitation, to an implementation of the method involving at least one computer and/or at least one computer network. The computer and/or computer network may comprise at least one processor which is configured for performing at least one of the method steps of the method according to the present disclosure. Specifically, each of the method steps may be performed by the computer and/or computer network. The method may be performed completely automatically, specifically without user interaction.

In a further aspect of the present disclosure, a method of determining at least one physiological parameter in a body tissue of a user is disclosed. The term “determining” as used herein is a broad term and is to be given its ordinary and customary meaning to a person of ordinary skill in the art and is not to be limited to a special or customized meaning. The term specifically may refer, without limitation, to a process of generating at least one representative result, in particular, by evaluating the at least one measurement signal as acquired by the transdermal sensor. The determining may specifically comprise at least one quantitative and/or qualitative detection of the physiological parameter in the body tissue of the user. For example, the physiological parameter, as outlined above, may comprise at least one analyte, e.g. blood glucose. Thus, in this example, the determination of the physiological parameter may comprise at least one analyte measurement of the analyte in the body tissue of the user.

The method comprises the following steps that, as an example, may be performed in the given order. It shall be noted, however, that a different order may generally also be possible. Further, it may also be possible to perform one or more of the method steps once or repeatedly. Further, it may also be possible to perform two or more of the method steps simultaneously or in a timely overlapping fashion. The method may comprise further method steps that are not listed.

The method comprises determining the physiological parameter by evaluating at least one detector signal provided by at least one transdermal sensor of at least one medical device according to the present disclosure, such as according to any one of the embodiments disclosed above and/or according to any one of the embodiments disclosed in further detail below. The determining of the physiological parameter comprises taking into account at least one bleeding detector signal provided by the bleeding detector of the body mount of the medical device.

Specifically, in case no bleeding is detected, the determining of the physiological parameter may comprise performing at least one initial calibration, specifically at least one initial background calibration, thereby obtaining at least one item of initial calibration. The term “calibration” as used herein is a broad term and is to be given its ordinary and customary meaning to a person of ordinary skill in the art and is not to be limited to a special or customized meaning. The term specifically may refer, without limitation, to at least one process for ensuring pre-defined or pre-specified measurement conditions and/or adjusting and/or adapting measurement conditions dependent on the medical device and/or hardware configurations of the medical device. Consequently, the term “initial calibration” as used herein is a broad term and is to be given its ordinary and customary meaning to a person of ordinary skill in the art and is not to be limited to a special or customized meaning. The term specifically may refer, without limitation, to at least one calibration being performed by the user upon first usage of the medical device. The result of the initial calibration may comprise the item of initial calibration which, specifically, can be used to transform or adapt the determined physiological parameter into a physiological parameter which would be obtained under pre-defined or pre-specified measurement conditions. The item of initial calibration may specifically be a device-specific item of calibration. The determining of the physiological parameter may comprise taking into account the item of initial calibration.

Alternatively, in case bleeding is detected, the determining of the physiological parameter may comprise taking into account at least one item of factory calibration. The term “factory calibration” as used herein is a broad term and is to be given its ordinary and customary meaning to a person of ordinary skill in the art and is not to be limited to a special or customized meaning. The term specifically may refer, without limitation, to at least one calibration process being performed by a manufacturer of the medical device, e.g. in a factory before, during and/or after assembly of the medical device. The result of the factory calibration may comprise the item of factory calibration which, specifically, can be used to transform or adapt the determined physiological parameter into a physiological parameter which would be obtained under pre-defined or pre-specified measurement conditions. The item of factory calibration may specifically be a device-independent item of calibration.

The item of factory calibration may be taken into account for a predetermined period of time after insertion of the intracorporal part of the transdermal medical device into the body tissue of the user. The predetermined period of time may be seven days, specifically five days, more specifically two days, more specifically 24 hours, more specifically 20 hours.

Additionally, or alternatively, after expiry of the predetermined period of time after insertion of the intracorporal part of the transdermal medical device into the body tissue of the user, the determining of the physiological parameter may comprise performing the at least one initial calibration, specifically the at least one initial background calibration, thereby obtaining the at least one item of initial calibration. The determining of the physiological parameter, specifically the further determining of the physiological parameter after the expiry of the predetermined period of time and after the initial calibration is performed, may comprise taking into account the item of initial calibration.

The method may be at least partially computer-implemented, specifically in above defined sense of “computer-implemented”.

In a further aspect of the present disclosure, a computer program is disclosed, comprising instructions which, when the program is executed by a computer or computer network, cause the computer or computer network to perform the method of monitoring a transdermal medical device according to the computer-implemented embodiment of the method of monitoring a transdermal medical device as disclosed herein.

Similarly, a computer-readable storage medium, specifically a non-transient computer-readable storage medium, is disclosed, comprising instructions which, when the instructions are executed by a computer or computer network, cause the computer or computer network to perform the method of monitoring a transdermal medical device according to the computer- implemented embodiment of the method of monitoring a transdermal medical device as disclosed herein.

As used herein, the term “computer-readable storage medium” specifically may refer to non-transitory data storage means, such as a hardware storage medium having stored thereon computer-executable instructions. The storage medium specifically may be or may comprise a storage medium such as a random-access memory (RAM) and/or a read-only memory (ROM).

In a further aspect of the present disclosure, a computer program is disclosed, comprising instructions which, when the program is executed by a computer or computer network, cause the computer or computer network to perform the method of determining at least one physiological parameter in a body tissue of a user according to the computer-implemented embodiment of the method of determining at least one physiological parameter in a body tissue of a user as disclosed herein.

Similarly, a computer-readable storage medium, specifically a non-transient computer-readable storage medium, is disclosed, comprising instructions which, when the instructions are executed by a computer or computer network, cause the computer or computer network to perform the method of determining at least one physiological parameter in a body tissue of a user according to the computer-implemented embodiment of the method of determining at least one physiological parameter in a body tissue of a user as disclosed herein.

Further disclosed and proposed herein is a computer program including computer-executable instructions for performing the method of monitoring a transdermal medical device and/or the method of determining at least one physiological parameter in a body tissue of a user according to the present disclosure in one or more of the embodiments enclosed herein when the instructions are executed on a computer or computer network. Specifically, the computer program may be stored on a computer-readable data carrier and/or on a computer-readable storage medium.

Thus, specifically, one, more than one, or even all of method steps of any one of the method of monitoring a transdermal medical device and/or of the method of determining at least one physiological parameter in a body tissue of a user as indicated above may be performed by using a computer or a computer network, preferably by using a computer program.

Further disclosed and proposed herein is a computer program product having program code means, in order to perform the method of monitoring a transdermal medical device and/or the method of determining at least one physiological parameter in a body tissue of a user according to the present disclosure in one or more of the embodiments enclosed herein when the program is executed on a computer or computer network. Specifically, the program code means may be stored on a computer-readable data carrier and/or on a computer-readable storage medium.

Further disclosed and proposed herein is a data carrier having a data structure stored thereon, which, after loading into a computer or computer network, such as into a working memory or main memory of the computer or computer network, may execute the method of monitoring a transdermal medical device and/or the method of determining at least one physiological parameter in a body tissue of a user according to one or more of the embodiments disclosed herein.

Further disclosed and proposed herein is a computer program product with program code means stored on a machine-readable carrier, in order to perform the method of monitoring a transdermal medical device and/or the method of determining at least one physiological parameter in a body tissue of a user according to one or more of the embodiments disclosed herein, when the program is executed on a computer or computer network. As used herein, a computer program product refers to the program as a tradable product. The product may generally exist in an arbitrary format, such as in a paper format, or on a computer-readable data carrier and/or on a computer-readable storage medium. Specifically, the computer program product may be distributed over a data network.

Finally, disclosed and proposed herein is a modulated data signal which contains instructions readable by a computer system or computer network, for performing the method of monitoring a transdermal medical device and/or the method of determining at least one physiological parameter in a body tissue of a user according to one or more of the embodiments disclosed herein.

Referring to the computer-implemented aspects of the disclosure, one or more of the method steps or even all of the method steps of the method of monitoring a transdermal medical device and/or of the method of determining at least one physiological parameter in a body tissue of a user according to one or more of the embodiments disclosed herein may be performed by using a computer or computer network. Thus, generally, any of the method steps including provision and/or manipulation of data may be performed by using a computer or computer network. Generally, these method steps may include any of the method steps, typically except for method steps requiring manual work, such as providing the samples and/or certain aspects of performing the actual measurements.

The devices and methods according to the present disclosure may provide a larger number of advantages over known devices and methods. Specifically, the body mount according to the present disclosure, specifically provided as the medical device and/or as the medical kit to the user, having the at least one capillary may allow bleeding detection which may occur upon insertion of the transdermal medical device, for example in the field of continuous blood glucose monitoring. The capillary may be provided in the base element, wherein, as an example, an opening of the capillary may be arranged adjacent to the through-hole, also referred to as “insertion site”, and, thus, in case of bleeding, may be configured for taking up the blood of the user. In the capillary, the blood may be detected by the bleeding detector, specifically using one or more of an optical bleeding detector or an electrical bleeding detector. Thus, by using the devices and methods according to the present disclosure, precise detection of bleeding for medical devices, such as for continuous glucose monitoring devices, may be possible.

The body mount according to the present disclosure specifically may provide a capillary channel in the base element, such as in a base element of a continuous glucose monitoring device, which, more specifically, may have an opening close to the insertion site of the transdermal medical device. After insertion of the transdermal medical device, once bleeding occurs, the blood may fill the capillary and may be detected by using the bleeding detector, for example by using an optical bleeding detector and/or an electrical bleeding detector. If the bleeding is detected after insertion, it may be possible to perform a complete fail-safe, to generate an item of information, e.g. a warning signal to the user regarding the measurement quality, and/or to initiate different calibration scenarios. For example, the blood detection may be also combined with further signals, for example impedance measurements for the signal correction. If no bleeding is detected, the medical device may perform an initial calibration, for example an initial background calibration, which specifically may increase the measurement precision. In case bleeding is detected, the medical device may use a factory calibration mode, which may be of lower precision, in particular during the first hours or days depending on the bleeding severity, but the user may be provided with information about the lower precision. It may be possible to combine both calibration modes. For example, if bleeding occurs, the initial calibration may be postponed for the predetermined period of time, which specifically depends on the bleeding severity. The medical device may use, during the predetermined period of time, the item of factory calibration. After expiry of the predetermined period of time and/or once the blood deposited at the transdermal medical device is reduced by the body, the medical device may perform the initial calibration and the medical device may use the determined item of initial calibration for further determination of the physiological parameter in order to improve the precision of the measurement. Additionally, or alternatively, since the bleeding severity often correlates with the signal distortion extent and duration, the medical device may be configured for detecting several levels of the bleeding, for example by using a plurality of bleeding detectors. Additionally, or alternatively, the medical device may be a continuous glucose monitoring device and may comprise a single use blood glucose detection mechanism built in the capillary.

Further, if the bleeding occurs upon insertion of the transdermal medical device into the body tissue of the user, the blood may fill an area around the body mount, specifically reaching an opening of the capillary. The capillary may be formed in the base element, specifically integrally during the manufacturing process, e.g. using an injection molding process. The base element may preferably made of polycarbonate. The capillary may be sealed by at least one foil, specifically at least one hydrophilic foil, for example being arranged at recess forming capillary walls. Thus, the capillary may be complete. Additionally, or alternatively, the capillary may be formed on any other desired way, e.g. completely formed in the base element. The capillary may be optionally coated by a hydrophilization layer, thus improving its wetting properties by the blood. A volume of the capillary may be chosen in a wide range and depends on a desired sensitivity for the bleeding. For example, larger volumes may require more blood to fill the capillary and, thus, lower sensitivities are achieved. Vice versa, smaller volumes may achieve higher sensitivities. The volume of the capillary may be defined as the volume which has to be filed by the blood to be detectable by the bleeding detector. Additionally, or alternatively, the body mount may comprise a plurality of bleeding detectors arranged along the capillary and, thus, may have several sensitivity levels. The capillary may be of any possible shape, such as curved, straight, with varied profile, e.g. thinner at the opening and widening from the opening along the capillary. Thus, it may be possible to combine high sensitivity levels for small blood volumes with the ability to detect larger blood volumes.

For example, the bleeding detector may comprise at least one optical bleeding detector. In order to optically detect the blood which fills the capillary, a beam of light may be sent through the capillary and may be detected by a photodetector. The wavelength and/or the spectrum of the light used may be chosen for maximum absorbance by the blood. For example, an optical emitter element comprising at least one light-emitting diode (LED) may be used. Further, a photodiode may be used for the detection of light. The optical bleeding detector may comprise a plurality of such LED and photodiode pairs. However, it may also be possible to use one LED and one or more photodiodes. The optical bleeding detector may be arranged for direct illumination. In this case, the optical emitter element and the optical detection element may be placed on opposing side of the capillary, specifically such that the light beam crosses the capillary. Additionally, or alternatively, the optical bleeding detector may be arranged for reflective illumination. In this case, the light beam may be reflected at least once at the capillary. The illumination path may specifically be defined by the shape of the capillary. In the reflective illumination arrangement, the optical emitter element and the optical detection element may be close to each other, e.g. on the control unit which may be mounted above the capillary. The capillary may be formed in the base element, which specifically may be designed to optimize the propagation of the light beam from the optical emitter element to the optical detection element. By using the plurality of optical detectors arranged along the capillary, several levels of bleeding may be detectable.

For example, the bleeding detector may comprise at least one electrical bleeding detector. The electrical bleeding detector may comprise at least two electrodes. The electrical bleeding detector may be configured for a conductivity measurement, such as by determining a resistance between the electrodes. It may also be possible to arrange a plurality of electrodes to be integrated along the capillary, for example being integrated into the capillary, in order to detect several levels of bleeding. Alternatively, the two electrodes may be arranged along the whole capillary, and the filling level may be determined by a drop in the resistance. Alternatively, one electrode may be also sufficient, specifically in case the function of the second electrode may be taken by one of the electrodes on an inserted transdermal sensor.

Additionally, or alternatively, the bleeding may also be detected manually, e.g. using bare eyes. In this case, the capillary may be designed such that the user may be able to see an indicator area, which e.g. turns distinguishable red. In the manual mode, the medical device, specifically the continuous glucose monitoring device, may ask the user whether bleeding occurred. In order to simplify the detection by the user, a green LED may be placed behind the detection area. If the capillary is not filled by the blood, the green light may be visible; otherwise, the red blood may absorb the green LED light and the user may see no light.

Summarizing and without excluding further possible embodiments, the following embodiments may be envisaged:

Embodiment 1: A body mount for mounting at least one transdermal medical device to a body surface of a user, comprising:
at least one base element having at least one mounting surface for attachment to the body surface of the user,
at least one capillary for taking up blood from the body of the user when the base element is attached to the body surface of the user;
at least one bleeding detector configured for detecting blood in the capillary.

Embodiment 2: The body mount according to the preceding embodiment, wherein the capillary is at least partially formed within the base element, specifically integrally.

Embodiment 3: The body mount according to the any one of the preceding embodiments, wherein the capillary is coated by at least one hydrophilization layer.

Embodiment 4: The body mount according to any one of the preceding embodiments, wherein the capillary is sealed by at least one foil, specifically at least one hydrophilic foil.

Embodiment 5: The body mount according to any one of the preceding embodiments, wherein the capillary comprises at least one capillary selected from the group consisting of: a straight capillary; a curved capillary; a conical capillary; a capillary with varying profile; a broadening capillary, specifically a capillary broadening from an opening towards an end of the capillary; a capillary formed in the adhesive of the mounting surface.

Embodiment 6: The body mount according to any one of the preceding embodiments, wherein the base element comprises at least one through-hole allowing the transdermal medical device to at least partially extend through the base element.

Embodiment 7: The body mount according to the preceding embodiment, wherein the capillary, specifically an opening of the capillary, is arranged adjacent to the through-hole.

Embodiment 8: The body mount according to any one of the preceding embodiments, wherein the bleeding detector is selected from the list comprising an optical bleeding detector and an electrical bleeding detector.

Embodiment 9: The body mount according to any one of the preceding embodiments, wherein the bleeding detector comprises at least one optical emitter element for emitting light into the capillary and at least one optical detection element for detecting light from the capillary.

Embodiment 10: The body mount according to the preceding embodiment, wherein the bleeding detector is configured for bleeding detection by at least one of a transmissive optical measurement and a reflective optical measurement.

Embodiment 11: The body mount according to any one of the preceding embodiments, wherein the base element is at least partially made of at least one optically transparent material, specifically wherein the capillary is at least partially integrated into the base element and wherein at least one wall of the capillary is at least partially made of the optically transparent material.

Embodiment 12: The body mount according to any one of the preceding embodiments, wherein the base element is at least partially made of at least one flexible material.

Embodiment 13: The body mount according to any one of the preceding embodiments, wherein the base clement is at least partially made of at least one plaster, specifically at least one adhesive plaster, more specifically at least one flexible plaster adapted for adhesion on the skin of the user.

Embodiment 14: The body mount according to any one of the preceding embodiments, wherein the mounting surface comprises at least one adhesive for adhesion to the body surface of the user.

Embodiment 15: The body mount according to any one of the preceding embodiments, wherein the bleeding detector comprises at least one electrical bleeding detector, wherein the electrical bleeding detector comprises at least two electrodes.

Embodiment 16: The body mount according to the preceding embodiment, wherein the electrical bleeding detector is configured for detection of blood by at least one of a conductivity measurement, a capacitive measurement and an impedance measurement.

Embodiment 17: The body mount according to any one of the preceding embodiments, wherein the body mount comprises a plurality of bleeding detectors arranged along the capillary in order to detect a filling level of the capillary.

Embodiment 18: The body mount according to any one of the preceding embodiments, further comprising at least one control unit, the control unit being configured for processing at least one bleeding detector signal from the bleeding detector.

Embodiment 19: The body mount according to the preceding embodiment, wherein the control unit is configured for generating at least one item of information regarding a bleeding status, specifically at least one warning signal in case a bleeding is detected.

Embodiment 20: A medical device, comprising at least one body mount according to any one of the preceding embodiments, the medical device further comprising at least one transdermal medical device having at least one intracorporal part being configured for transdermal insertion into a body tissue of the user and at least one extracorporal part attached to the body mount.

Embodiment 21: The medical device according to the preceding embodiment, wherein the transdermal medical device is selected from the group consisting of: a transdermal sensor, specifically at least one transdermal sensor for measuring at least one physiological parameter, more specifically a transdermal analyte sensor, more specifically at least one transdermal analyte sensor for detecting at least analyte in a bodily fluid of the user; an infusion device having at least one tubular infusion element for administering at least one medically active substance into the body tissue of the user.

Embodiment 22: The medical device according to the preceding embodiment, wherein the transdermal medical device comprises at least one transdermal sensor, wherein the medical device further comprises at least one evaluation unit, wherein the evaluation unit is configured for determining at least one physiological parameter by evaluating at least one detector signal provided by the transdermal sensor, wherein the evaluation unit is further configured for taking into account at least one bleeding detector signal provided by the bleeding detector.

Embodiment 23: A medical kit comprising at least one medical device according to any one of the preceding embodiments referring to a medical device, further comprising at least one insertion aid for inserting the intracorporal part of the transdermal medical device into the body tissue of the user.

Embodiment 24: A method of monitoring a transdermal medical device, the method comprising evaluating at least one bleeding detector signal of at least one bleeding detector of at least one medical device according to any one of the preceding embodiments referring to a medical device for detecting blood in the least one capillary of the body mount of the medical device.

Embodiment 25: The method according to the preceding embodiment, wherein the body mount comprises at least one control unit, wherein the method further comprises processing the at least one bleeding detector signal from the bleeding detector by using the control unit.

Embodiment 26: The method according to the preceding embodiment, wherein the processing of the bleeding detector signal comprises generating at least one item of information regarding a bleeding status, specifically at least one warning signal in case a bleeding is detected.

Embodiment 27: The method according to any one of the preceding method embodiments, wherein the method is at least partially computer-implemented.

Embodiment 28: A method of determining at least one physiological parameter in a body tissue of a user, the method comprising determining the physiological parameter by evaluating at least one detector signal provided by at least one transdermal sensor of at least one medical device according to any one of the preceding embodiments referring to a medical device, wherein the determining of the physiological parameter comprises taking into account at least one bleeding detector signal provided by the bleeding detector of the body mount of the medical device.

Embodiment 29: The method according to the preceding embodiment, wherein, in case no bleeding is detected, the determining of the physiological parameter comprises performing at least one initial calibration, specifically at least one initial background calibration, thereby obtaining at least one item of initial calibration.

Embodiment 30: The method according to the preceding embodiment, wherein the determining of the physiological parameter comprises taking into account the item of initial calibration.

Embodiment 31: The method according to any one of the three preceding embodiments, wherein, in case bleeding is detected, the determining of the physiological parameter comprises taking into account at least one item of factory calibration.

Embodiment 32: The method according to the preceding embodiment, wherein the item of factory calibration is taken into account for a predetermined period of time after insertion of the intracorporal part of the transdermal medical device into the body tissue of the user.

Embodiment 33: The method according to the preceding embodiment, wherein the predetermined period of time is seven days, specifically five days, more specifically two days, more specifically 24 hours, more specifically 20 hours.

Embodiment 34: The method according to any one of the two preceding embodiments, wherein, after expiry of the predetermined period of time after insertion of the intracorporal part of the transdermal medical device into the body tissue of the user, the determining of the physiological parameter comprises performing at least one initial calibration, specifically at least one initial background calibration, thereby obtaining at least one item of initial calibration.

Embodiment 35: The method according to the preceding embodiment, wherein the determining of the physiological parameter comprises taking into account the item of initial calibration.

Embodiment 36: The method according to any one of the eight preceding embodiments, wherein the method is at least partially computer-implemented.

Embodiment 37: A computer program comprising instructions which, when the program is executed by a computer or computer network, cause the computer or computer network to perform the method of monitoring a transdermal medical device according to embodiment 27.

Embodiment 38: A computer-readable storage medium, specifically a non-transient computer-readable storage medium, comprising instructions which, when the instructions are executed by a computer or computer network, cause the computer or computer network to perform the method of monitoring a transdermal medical device according to embodiment 27.

Embodiment 39: A computer program comprising instructions which, when the program is executed by a computer or computer network, cause the computer or computer network to perform the method of determining at least one physiological parameter in a body tissue of a user according to embodiment 36.

Embodiment 40: A computer-readable storamedium, specifically a non-transient computer-readable storage medium, comprising instructions which, when the instructions are executed by a computer or computer network, cause the computer or computer network to perform the method of determining at least one physiological parameter in a body tissue of a user according to embodiment 36.

### BRIEF DESCRIPTION OF THE DRAWINGS

Further optional features and embodiments will be disclosed in more detail in the subsequent description of embodiments, preferably in conjunction with the dependent claims. Therein, the respective optional features may be realized in an isolated fashion as well as in any arbitrary feasible combination, as the skilled person will realize. The scope of the invention is not restricted by the preferred embodiments. The embodiments are schematically depicted in the Figures. Therein, identical reference numbers in these Figures refer to identical or functionally comparable elements.

In the Figures:

FIGS. 1A and 1B show experimental data of a continuous glucose monitoring device;

FIG. 2 shows an embodiment of a medical device in a perspective view;

FIG. 3 shows an embodiment of a medical kit in a schematic view;

FIGS. 4A, 4B and 4C show different embodiments of a body mount with an optical bleeding detector in a schematic view;

FIG. 4D shows a diagram of a molar extinction coefficient as a function of wavelength;

FIGS. 5A and 5B show an embodiment of a body mount with an electrical bleeding detector in a schematic view;

FIG. 6 shows a flow chart of an embodiment of a method of monitoring a transdermal medical device;

FIG. 7 shows a flow chart of an embodiment of a method of determining at least one physiological parameter in a body tissue of a user; and

FIG. 8 shows an embodiment of a capillary of the medical device.

Although the exemplification set out herein illustrates embodiments of the invention in several forms, the embodiments disclosed below are not intended to be exhaustive or to be construed as limiting the scope of the invention to the precise forms disclosed.

### DETAILED DESCRIPTION

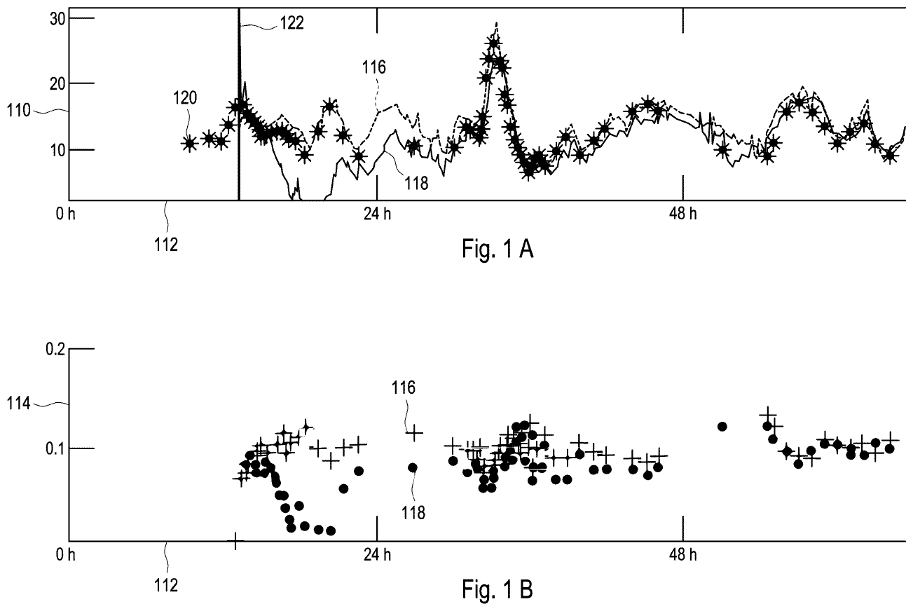

FIGS. 1A and 1B show experimental data of a continuous glucose monitoring (CGM) device, specifically of an amperometric CGM device. These experimental data were not acquired by using a medical device according to the present disclosure, but with known CGM devices. In the diagram of FIG. 1A, a current 110 in nA acquired by the continuous glucose monitoring device is shown as a function of time 112. In the diagram of FIG. 1B, a sensitivity 114 in nA/mg/dl of the CGM device is shown as a function of time 112. FIG. 1A shows two curves of the current 110 simultaneously acquired with two different CGM devices at the same user (denoted by reference numbers 116 and 118). Additionally, FIG. 1A shows the corresponding blood glucose concentration (denoted by reference number 120). The sensitivity 114 in FIG. 1B was calculated as the quotient of the current 110 and the blood glucose concentration 120 for both CGM devices 116, 118. The CGM device 118 caused bleeding upon insertion of the sensor. The time of insertion is marked in FIG. 1A by line 122. As can be seen in FIG. 1B, the bleeding effect clearly reduces the current 110 during the first 20 hours. The corresponding sensitivity 114 was also strongly reduced from the average value of 0.1 nA/mg/dl.

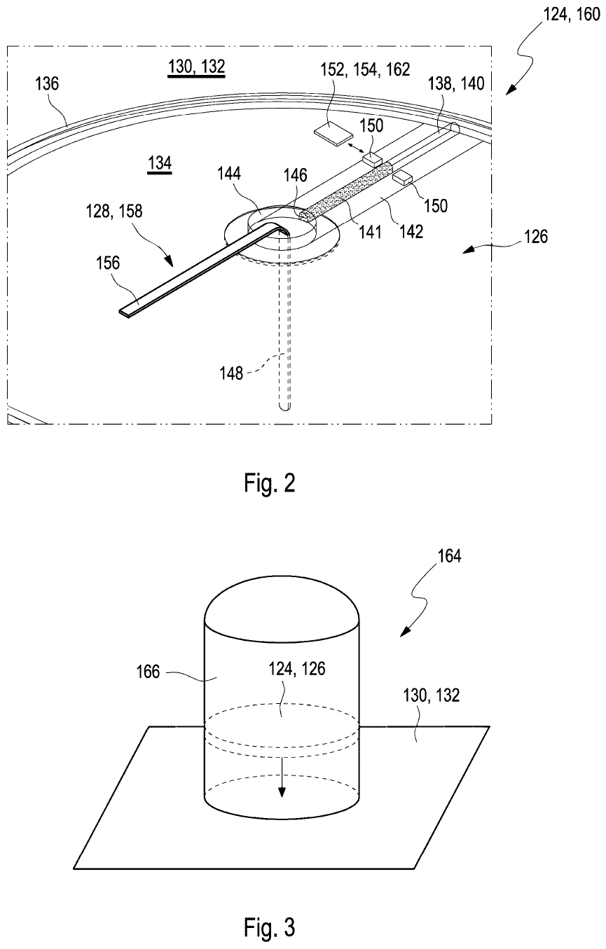

FIG. 2 shows an exemplary embodiment of a medical device 124 according to the present disclosure in a perspective view. The medical device 124 comprises at least one body mount 126 for mounting at least one transdermal medical device 128 to a body surface 130 of a user 132.

The body mount 126 comprises at least one base element 134 having at least one mounting surface 136 for attachment to the body surface 130 of the user 132. The base element 134 may at least partially be made of at least one flexible material. Additionally, or alternatively, the base element 134 may be at least partially made of at least one plaster (not shown in FIG. 2), specifically at least one adhesive plaster, more specifically at least one flexible plaster adapted for adhesion on the skin of the user 132. Specifically, the mounting surface 136 may comprise at least one adhesive for adhesion to the body surface 130 of the user 132.

The body mount 126 further comprises at least one capillary 138 for taking up blood 141 from the body of the user 132 when the base element 134 is attached to the body surface 130 of the user 132. The capillary 138 may be at least partially formed within the base element 134, specifically integrally. In the example of FIG. 2, the capillary 138 may be at least partially formed by at least one recess 140 in the base element 134, e.g. during a molding manufacturing process. Additionally, the capillary 138 may be sealed by at least one foil 142, specifically at least one hydrophilic foil.

As can be seen in FIG. 2, the base element 134 may further comprise at least one through-hole 144 allowing the transdermal medical device 128 to at least partially extend through the base element 134. The capillary 138, specifically an opening 146 of the capillary 138, may be arranged adjacent to the through-hole 144. Thus, in case insertion of at least one intracorporal part 148 of the transdermal medical device 128 may cause bleeding in the body tissue of the user 132, the opening 146 of the capillary 138 may allow the blood 141 to enter the capillary 138.

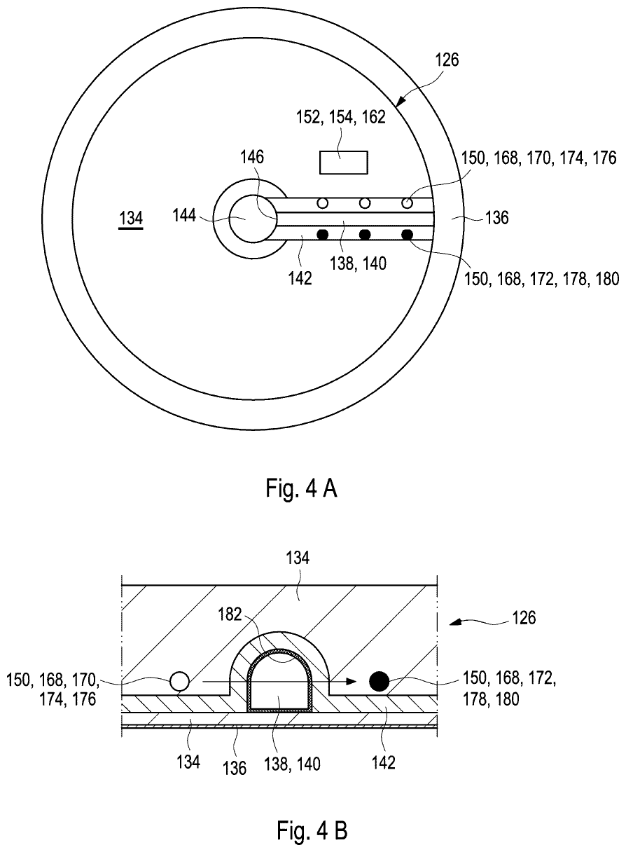

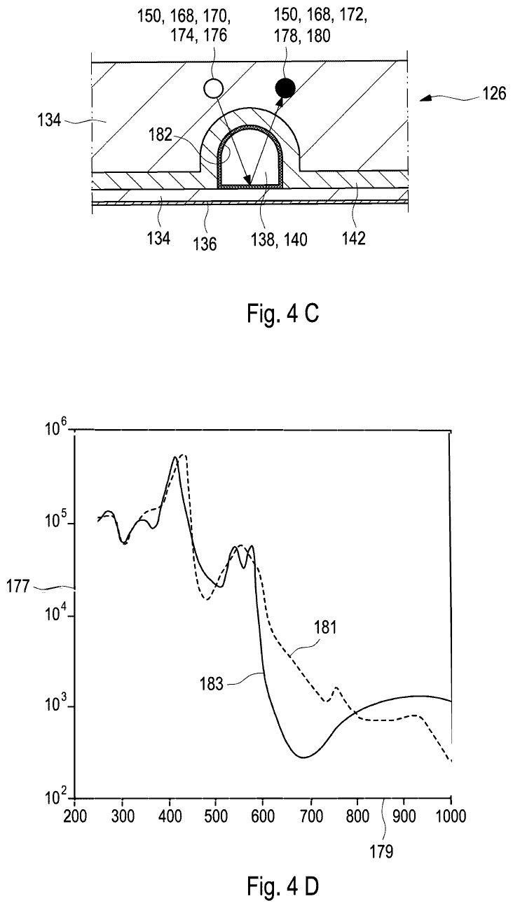

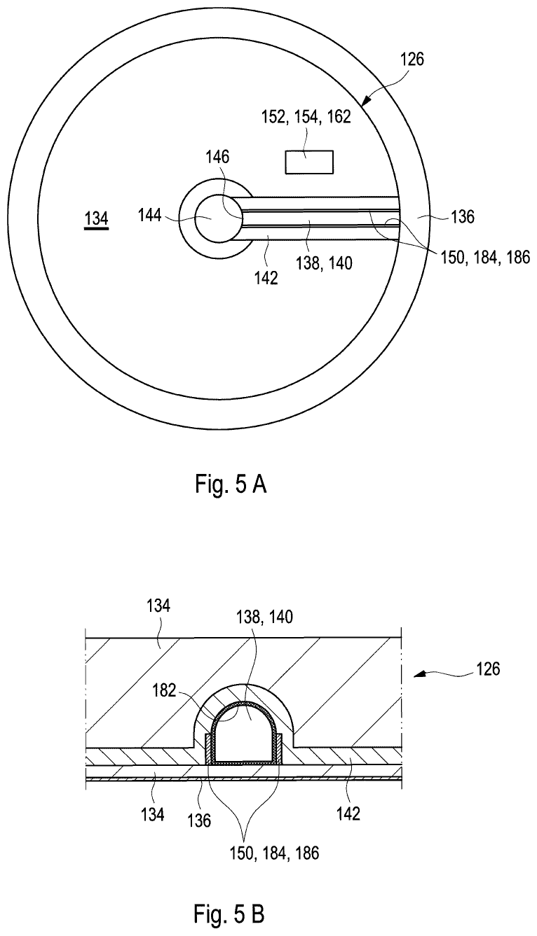

The body mount 126 further comprises at least one bleeding detector 150 configured for detecting blood 141 in the capillary 138. Possible embodiments of the bleeding detector 150 are shown in FIGS. 4A to 5B and will be described in further detail below. Thus, for a detailed description of the bleeding detector 150, reference is made to the description of FIGS. 4A to 5B.

The body mount 126 may further comprise at least one control unit 152. The control unit 152 may specifically comprise one or more processors 154. As shown in FIG. 2, the control unit 152 may be, directly or indirectly, connected to the bleeding detector 150. The control unit 152 specifically may be configured, such as by software programming, for processing at least one bleeding detector signal from the bleeding detector 150. For example, the control unit 152 may be configured for generating at least one item of information regarding a bleeding status, specifically at least one warning signal in case a bleeding is detected.

The medical device 124 further comprising the at least one transdermal medical device 128 having the at least one intracorporal part 148 being configured for transdermal insertion into a body tissue of the user 132 and at least one extracorporal part 156 attached to the body mount 126. In the example of FIG. 2, the transdermal medical device 128 may comprise at least one transdermal sensor 158, specifically at least one transdermal analyte sensor for detecting, e.g. continuously detecting, at least analyte in a bodily fluid of the user 132. Thus, in this example, the medical device 124 may be a continuous glucose monitoring (CGM) device 160. However, other types of transdermal medical devices 128 are also feasible, for example an infusion device (not shown in FIG. 2) having at least one tubular infusion element for administering at least one medically active substance into the body tissue of the user 132.

The medical device 124 may further comprise at least one evaluation unit 162. In the exemplary embodiment of FIG. 2, the evaluation unit 162 may specifically be identical with the control unit 152, such as by being completely embodied by the same logic circuitry. The evaluation unit 162 may be configured, such as by software programming, for determining the at least one physiological parameter by evaluating the at least one detector signal provided by the transdermal sensor 158, specifically by the transdermal analyte sensor. The evaluation unit 162 may further be configured, such as by software programming, for taking into account the at least one bleeding detector signal provided by the bleeding detector 150. The bleeding detector 150 may be configured for directly or indirectly providing the bleeding detector signal to the evaluation unit 162.

FIG. 3 shows an embodiment of a medical kit 164 in a schematic view. The medical kit 164 comprises at least one medical device 124 according to the present disclosure, such as according to the exemplary embodiment of FIG. 2 and/or according to any other embodiments disclosed herein. Thus, for a description of the medical device 124, reference is made to the description of FIG. 2. As schematically shown in FIG. 3, the medical kit 164 further comprises at least one insertion aid 166 for inserting the intracorporal part 148 of the transdermal medical device 128 into the body tissue of the user 132. Specifically, upon inserting the intracorporal part 148 of the transdermal medical device 128 into the body tissue of the user 132, the insertion aid 166 may be configured for attaching the base element 134 of the body mount 126 to the body surface 130 of the user 132.

In FIGS. 4A to 4C, different embodiments of the body mount 126 with an optical bleeding detector 168 are shown in a schematic view. Specifically, FIG. 4A shows a top view of the body mount 126, FIG. 4B shows a side view of a first embodiment of the optical bleeding detector 168 and FIG. 4C shows a side view of a second embodiment of the optical bleeding detector 168.

As shown in FIGS. 4A to 4C, the bleeding detector 150 may comprise at least one optical emitter element 170 for emitting light into the capillary 138 and at least one optical detection element 172 for detecting light from the capillary 138. The optical emitter element 170 may comprise at least one light source 174, such as a light emitting diode (LED) 176, for emitting light into the capillary 138. The optical detection element 172 may comprise at least one photosensitive element 178 configured for determining at least one optical parameter, such as an intensity and/or a power of light by which at least one sensitive area of the optical detection element 172 is irradiated. For example, the optical detection element 172 may comprise at least one photodiode 180. Additionally, the base element 134 may be at least partially made of at least one optically transparent material. Specifically, the capillary 138 may be at least partially integrated into the base element 134 and at least one wall of the capillary 138 may be at least partially made of the optically transparent material.

In the first embodiment of FIG. 4B, the optical emitter element 170 and the optical detection element 172 may be arranged for direct illumination. The optical emitter element 170 and the optical detection element 172 may be arranged on opposing side of the capillary 138, specifically such that light emitted by the optical emitter element 170 may directly illuminate the capillary 138 and, subsequently, the optical detection element 172, more specifically without being reflected along an illumination path. Thus, in this arrangement, the bleeding detector 150 may be configured for transmissive optical measurement.

Additionally, or alternatively, in the second embodiment of FIG. 4C, the optical emitter element 170 and the optical detection element 172 may be arranged for reflective illumination. The optical emitter element 170 and the optical detection element 172 may be arranged on the same side of the capillary 138, specifically adjacent to each other, such that light emitted by the optical emitter element 170 may illuminate the capillary 138, be reflected at least once at the capillary 138, e.g. at least once at one or more reflective capillary walls, and, subsequently, illuminate the optical detection element 172. The reflective illumination may specifically comprise at least one reflection of light in the illumination path. Thus, in this arrangement, the bleeding detector 150 may be configured for reflective optical measurement.

The body mount 126, as shown in FIG. 4A, may comprise a plurality of bleeding detectors 150 arranged along the capillary 138 in order to detect a filling level of the capillary 138. Specifically, the plurality of bleeding detectors 150 may comprise one or more optical bleeding detectors 168 according to any one of the first and/or second embodiment shown in FIGS. 4B and 4C.

Further, the capillary 138 may be coated by at least one hydrophilization layer 182, specifically in order to improve the capillary's 138 wetting properties.

FIG. 4D shows a diagram of a molar extinction coefficient 177 of blood 141 in cm⁻¹/M as a function of wavelength 179 in nm. Specifically, the molar extinction coefficient 177 of hemoglobin 181 and oxyhemoglobin 183 is shown as a function of wavelength 179. As outlined above, the bleeding detector 150, in the exemplary embodiments of FIGS. 4A to 4C, may comprise the optical emitter element 170 for emitting light into the capillary 138. The optical emitter element 170 may specifically be configured for emitting light in a spectral range where an absorbance of blood 141 is maximum, for example in a spectral range at least partially comprising wavelengths from 350 to 450 nm and/or wavelengths from 500 to 600 nm. As shown in the diagram of FIG. 4D, the molar extinction coefficient 177 of hemoglobin 181 and oxyhemoglobin 183 is at local maximum in the wavelength ranges from 350 to 450 nm and/or from 500 to 600 nm, and, thus, blood 141 can be effectively detected using the optical bleeding detector 170. However, other wavelength ranges are also feasible.

Additionally, or alternatively to the optical bleeding detector 168, the bleeding detector 150 may be an electrical bleeding detector 184. FIGS. 5A and 5B show an embodiment of the body mount 126 with the electrical bleeding detector 184 in a schematic view. The electrical bleeding detector 184 may comprise at least two electrodes 186. For example, the electrical bleeding detector 184 may comprise a plurality of electrodes 186 arranged along the capillary 138. For example, the electrical bleeding detector 184 may comprise two electrodes 186 extending along the capillary 138, specifically along the entire capillary 138. Additionally or alternatively, in case the transdermal medical device 128 comprises at least one transdermal sensor 158, at least one of the electrodes 186 of the electrical bleeding detector 184 may be formed by the transdermal sensor 158. The electrical bleeding detector 184 may be configured for detection of blood by at least one of a conductivity measurement, a capacitive measurement and an impedance measurement.

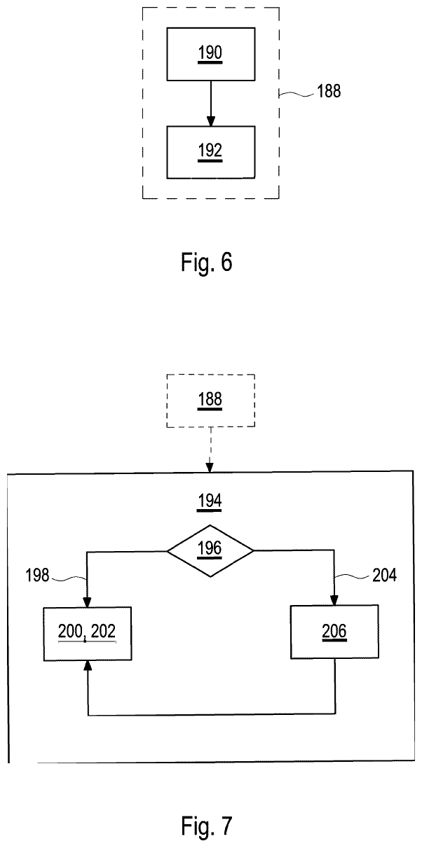

FIG. 6 shows a flow chart of an exemplary embodiment of a method of monitoring a transdermal medical device 128 (denoted by reference number 188). In the method of monitoring the transdermal medical device 128, the medical device 124 according to the present disclosure, such as according to the exemplary embodiment of FIG. 2, may be used. Thus, for a description of the medical device 124 to be used in the method, reference is made to the description of FIG. 2. Other embodiments are, however, also possible.

The method comprises the following steps that, as an example, may be performed in the given order. It shall be noted, however, that a different order may generally also be possible. Further, it may also be possible to perform one or more of the method steps once or repeatedly. Further, it may also be possible to perform two or more of the method steps simultaneously or in a timely overlapping fashion. The method may comprise further method steps that are not listed.

The method comprises evaluating the at least one bleeding detector signal of the at least one bleeding detector 150 of the at least one medical device 124 according to the present disclosure for detecting blood 141 in the least one capillary 138 of the body mount 126 of the medical device 124 (denoted by reference number 190).

The body mount 126 specifically may comprise the at least one control unit 152 as outlined above. The method may further comprise processing the at least one bleeding detector signal from the bleeding detector 150 by using the control unit 152 (denoted by reference number 192). The processing of the bleeding detector signal may comprise generating at least one item of information regarding a bleeding status, specifically at least one warning signal in case a bleeding is detected.

The method specifically may be at least partially computer-implemented. Thus, as an example, the method of FIG. 6 may at least partially be performed by the evaluation unit 162 of the medical device 124.

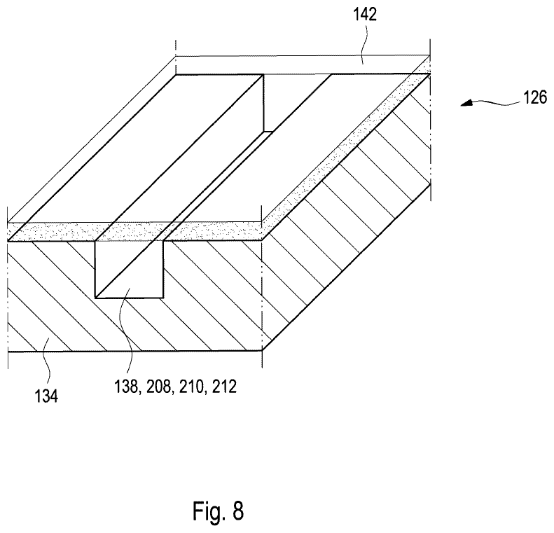

FIG. 7 shows a flow chart of an exemplary embodiment of a method of determining at least one physiological parameter in a body tissue of a user 132. In the method of determining at least one physiological parameter in a body tissue of a user 132, the medical device 124 according to the present disclosure, such as according to the exemplary embodiment of FIG. 2, may be used. Thus, for a description of the medical device 124 to be used in the method, reference is made to the description of FIG. 2. Other embodiments are, however, also possible. The method comprises the following steps that, as an example, may be performed in the given order. It shall be noted, however, that a different order may generally also be possible. Further, it may also be possible to perform one or more of the method steps once or repeatedly. Further, it may also be possible to perform two or more of the method steps simultaneously or in a timely overlapping fashion. The method may comprise further method steps that are not listed. The method comprises determining the physiological parameter by evaluating at least one detector signal provided by the at least one transdermal sensor 158 of the at least one medical device 124 according to the present disclosure (denoted by reference number 194). The determining of the physiological parameter comprises taking into account at least one bleeding detector signal provided by the bleeding detector 150 of the body mount 126 of the medical device 124. For example, as shown in FIG. 7, the method may comprise, prior to and/or at least partially simultaneously to the determining of the physiological parameter, monitoring the transdermal medical device 128, e.g. by performing the method of monitoring the transdermal medical device 128 according to the present disclosure (denoted by reference number 188).

Further, as can be seen in FIG. 7, the method may comprise a decision step (denoted by reference number 196). Specifically, in the decision step, it may be determined if bleeding occurred during insertion of the at least one intracorporal part 148 of the transdermal medical device 128 into the body tissue of the user 132. In case no bleeding is detected (denoted by reference number 198), the determining of the physiological parameter may comprise performing at least one initial calibration (denoted by reference number 200), specifically at least one initial background calibration, thereby obtaining at least one item of initial calibration. The determining of the physiological parameter may comprise taking into account the item of initial calibration (denoted by reference number 202).

Alternatively, in case bleeding is detected (denoted by reference number 204), the determining of the physiological parameter may comprise taking into account at least one item of factory calibration (denoted by reference number 206). The item of factory calibration may be taken into account for a predetermined period of time after insertion of the intracorporal part 148 of the transdermal medical device 128 into the body tissue of the user 132. The predetermined period of time may be seven days, specifically five days, more specifically two days, more specifically 24 hours, more specifically 20 hours.

Further, after expiry of the predetermined period of time after insertion of the intracorporal 148 part of the transdermal medical device 128 into the body tissue of the user 132, the determining of the physiological parameter may comprise performing the at least one initial calibration (denoted by reference number 200), specifically the at least one initial background calibration, thereby obtaining the at least one item of initial calibration. The determining of the physiological parameter may comprise taking into account the item of initial calibration (denoted by reference number 202).

FIG. 8 shows a further exemplary embodiment of the capillary 138 of the medical device 124. The exemplary embodiment of the capillary 138 of FIG. 8 may be used in any exemplary embodiment of the medical device 124 disclosed herein, such as the exemplary embodiment shown in FIG. 2, and/or in any exemplary embodiment of the body mount 126 disclosed herein, such as the exemplary embodiments shown in FIGS. 4A to 5B. In this example, as can be seen in FIG. 8, the capillary 138 may be at least partially formed within the base element 134 by providing the base element 134 with at least one recess 208, e.g. at least one groove 210. The capillary 138 may be sealed by the at least one foil 142, specifically at least one hydrophilic foil. Thus, in this example, the capillary 138 being sealed by the at least one foil 142 may form a complete capillary 212. The foil 142 may seal the recess 208 in the base element 134 to form the capillary 138.

### LIST OF REFERENCE NUMBERS

110 current
112 time
114 sensitivity
116 CGM device 1
118 CGM device 2
120 blood glucose concentration
122 time of insertion
124 medical device
126 body mount
128 transdermal medical device
130 body surface
132 user
134 base element
136 mounting surface
138 capillary
140 recess
141 blood
142 foil
144 through-hole
146 opening
148 intracorporal part
150 bleeding detector
152 control unit
154 processors
156 extracorporal part
158 transdermal sensor
160 CGM device
162 evaluation unit
164 medical kit
166 insertion aid
168 optical bleeding detector
170 optical emitter element
172 optical detection element
174 light source
176 LED
177 molar extinction coefficient
178 photosensitive element
179 wavelength
180 photodiode
181 molar extinction coefficient of hemoglobin
182 hydrophilization layer
183 molar extinction coefficient oxyhemoglobin
184 electrical bleeding detector
186 electrodes
188 monitoring a transdermal medical device
190 evaluating the bleeding detector signal
192 processing the bleeding detector signal
194 determining the physiological parameter
196 decision step
198 no bleeding detected
200 initial calibration
202 comprise taking into account the item of initial calibration
204 bleeding detected
206 taking into account the item of factory calibration
208 recess
210 groove
212 complete capillary

While this invention has been described as having an exemplary design, the present invention may be further modified within the spirit and scope of this disclosure. This application is therefore intended to cover any variations, uses, or adaptations of the invention using its general principles.

What is claimed is:

1. A body mount for mounting at least one transdermal medical device to a body surface of a user, comprising:
at least one base element having at least one mounting surface adapted to be attached to the body surface of the user;
at least one capillary adapted to take up blood from the body of the user when the base element is attached to the body surface of the user; and
at least one bleeding detector configured to detect blood in the capillary.

2. The body mount according to claim 1, wherein the capillary is at least partially formed within the base element.

3. The body mount according to claim 1, wherein the capillary is sealed by at least one foil.

4. The body mount according to claim 1, wherein the bleeding detector comprises at least one optical emitter element for emitting light into the capillary and at least one optical detection element for detecting light from the capillary.

5. The body mount according to claim 1, wherein the base element is at least partially made of at least one optically transparent material, wherein the capillary is at least partially integrated into the base element and wherein at least one wall of the capillary is at least partially made of the optically transparent material.

6. The body mount according to claim 1, wherein the base element is at least partially made of at least one plaster.

7. The body mount according to claim 1, wherein the mounting surface comprises at least one adhesive for adhesion to the body surface of the user.

8. The body mount according to claim 1, wherein the bleeding detector comprises at least one electrical bleeding detector, wherein the electrical bleeding detector comprises at least two electrodes, wherein the electrical bleeding detector is configured for detection of blood by at least one of a conductivity measurement, a capacitive measurement and an impedance measurement.

9. The body mount according to claim 1, further comprising at least one control unit, the control unit being configured for processing at least one bleeding detector signal from the bleeding detector, wherein the control unit is configured for generating at least one item of information regarding a bleeding status.

10. A medical device, comprising the body mount according to claim 1, the medical device further comprising at least one transdermal medical device having at least one intracorporal part being configured for transdermal insertion into a body tissue of the user and at least one extracorporal part attached to the body mount.

11. The medical device according to claim 10, wherein the transdermal medical device comprises at least one transdermal sensor, wherein the medical device further comprises at least one evaluation unit, wherein the evaluation unit is configured for determining at least one physiological parameter by evaluating at least one detector signal provided by the transdermal sensor, wherein the evaluation unit is further configured for taking into account at least one bleeding detector signal provided by the bleeding detector.

12. A medical kit comprising the medical device according to claim 10, further comprising at least one insertion aid for inserting the intracorporal part of the transdermal medical device into the body tissue of the user.

13. A method of monitoring a transdermal medical device, the method comprising evaluating at least one bleeding detector signal of at least one bleeding detector of at least one medical device according to claim 10 for detecting blood in the least one capillary of the body mount of the medical device.

14. A method of determining at least one physiological parameter in a body tissue of a user, the method comprising determining the physiological parameter by evaluating at least one detector signal provided by at least one transdermal sensor of the medical device according to claim 10, wherein the determining of the physiological parameter comprises taking into account at least one bleeding detector signal provided by the bleeding detector of the body mount of the medical device.

15. A non-transient computer-readable storage medium, comprising instructions which, when the instructions are executed by a computer or computer network, cause the computer or computer network to perform the method of monitoring a transdermal medical device according to claim 13.

16. A non-transient computer-readable storage medium, comprising instructions which, when the instructions are executed by a computer or computer network, cause the computer or computer network to perform the method of determining at least one physiological parameter in a body tissue of a user according to claim 14.
